(19) United States
(12) Patent Application Publication
(10) Pub. No.: US 2023/0240876 A1
(43) Pub. Date: Aug. 3, 2023

(54) Brace for Posterior Tibial Tendon Dysfunction

(71) Applicant: Medical Specialties, Inc., Charlotte, NC (US)

(72) Inventor: Eric Lee Gaylord, Matthews, NC (US)

(21) Appl. No.: 18/133,694

(22) Filed: Apr. 12, 2023

Related U.S. Application Data
(63) Continuation of application No. 16/032,274, filed on Jul. 11, 2018.
(60) Provisional application No. 62/531,476, filed on Jul. 12, 2017.
(60) Provisional application No. 62/596,388, filed on Dec. 8, 2017.

(51) Int. Cl. A61F 5/01; A61F 5/30

(52) U.S. Cl. CPC A61F 5/0111; A61F 5/30; A61F 2005/0172

(57) ABSTRACT

The present arch-support brace includes an advantageous strapping configuration that alleviates the painful symptoms of posterior tibial tendon dysfunction by applying consistent compression to the patient's foot, ankle, and lower leg. The arch-support brace includes a tibial-tendon support strap and a repositionable arch pad that lifts and rolls the patient's arch and inwardly rolls the patient's heel.

### CROSS-REFERENCE TO PRIORITY APPLICATIONS

This application is a continuation of U.S. patent application Ser. No. 16/032,274 for a Brace for Posterior Tibial Tendon Dysfunction (filed Jul. 11, 2018, and published Jan. 17, 2019, as U.S. Patent Application Publication No. US2019/0015234 A1), which claims the benefit of both U.S. Patent Application No. 62/531,476 for a Brace for Posterior Tibial Tendon Dysfunction (filed Jul. 12, 2017) and U.S. Patent Application No. 62/596,388 for a Brace for Posterior Tibial Tendon Dysfunction (filed Dec. 8, 2017). Each of the foregoing commonly assigned patent applications and patent application publication is hereby incorporated by reference in its entirety.

### FIELD OF THE INVENTION

The present invention relates to an arch-support brace that helps alleviate the painful symptoms of posterior tibial tendon dysfunction.

### BACKGROUND

The posterior tibial tendon is a foot-supporting tendon that connects the bones in the inner region of the foot to the calf muscle. Tearing or inflammation of the posterior tibial tendon, which defines the arch of the human foot, is referred to as “posterior tibial tendon dysfunction” or “PTTD.” This syndrome initially causes painful swelling at the inside of the foot and ankle. Posterior tibial tendon dysfunction typically leads to a fallen foot arch or “flatfoot,” which is often characterized not only by the fallen foot arch but also by an inward rolling of the foot and an associated outward heal tilt. Over time, the condition worsens and the pain extends to the outside of the ankle.

Posterior tibial tendon dysfunction is often caused by tendon overuse through physical exertion or strain (e.g., such as resulting from injury or obesity), and typically worsens with physical activity. Posterior tibial tendon dysfunction causes limited mobility and chronic pain. If braces or orthotics (i.e., shoe inserts) fail to provide relief, surgery may be required to alleviate the PTTD symptoms. Foot surgery, however, is complicated and patient recovery is slow.

### SUMMARY

In one aspect, the present arch-support braces for posterior tibial tendon dysfunction (i.e., “PTTD”) include a tibial-tendon support strap associated with an ankle support sleeve (e.g., attached to or attachable to an ankle support sleeve). The tibial-tendon support strap is configured (i) to extend at least partially around (e.g., to encircle) a patient's foot (e.g., via an under-and-over wrapping) and (ii) to apply a force to assist (e.g., reduce strain on) the patient's tibial tendon. The arch-support braces may include an arch pad contoured to correspond to the patient's mid-foot arch (e.g., the medial arch). Typically, the arch pad is moveably connected to a tibial-tendon support strap to facilitate repositioning along the length of the tibial-tendon support strap. The tibial-tendon support strap may be configured so that tension in the tibial-tendon support strap provides a force that inverts the patient's calcaneus to help restore proper alignment. The present arch-support braces help alleviate the painful symptoms of posterior tibial tendon dysfunction, and an aspect of this disclosure is the application and use of arch-support braces configured to address structural consequences of posterior tibial tendon dysfunction (i.e., “PTTD”) involving both arch drop and heal rotation.

In another aspect, the present arch-support braces for posterior tibial tendon dysfunction (i.e., “PTTD”) optionally include an advantageous lacing configuration that provides support for a patient's lower leg, ankle, and foot. By pulling an improved lacing mechanism, tension in a lace equilibrates along the length of the arch-support braces in a way that applies consistent compression to the patient's lower leg and ankle.

Exemplary arch-support braces (e.g., PTTD-brace splints) include an ankle support sleeve and a connecting front-panel tongue (e.g., a stretchable tongue) that together form an ankle-brace boot, which is securely positioned around a patient's ankle and lower leg. A lace, which typically resists excessive stretching, is threaded through lace-redirection mechanisms (e.g., eyelets) positioned on the ankle support sleeve (e.g., along the ankle support sleeve's free front edges) and through a lacing closure tab. Regardless of the shape and girth of the patient's lower leg, pulling the lacing closure tab circumferentially around the ankle-brace boot, and then releasably securing the lacing closure tab to the ankle-brace boot, can help achieve cast-like compression of the arch-support braces to the patient's lower leg and ankle.

The foregoing illustrative summary, as well as other exemplary objectives and/or advantages of the invention, and the manner in which the same are accomplished, are further explained within the following detailed description and its accompanying drawings and photographs.

### BRIEF DESCRIPTION OF THE DRAWINGS

FIG. 1 is a front perspective view of an exemplary PTTD-brace embodiment including an arch pad and tibial-tendon support strap, wherein one or more of the other straps depicted in FIG. 1 may be optional.

FIG. 2 is a side elevation view of the PTTD-brace of FIG. 1, wherein FIG. 2 schematically depicts that the arch pad may be movably mounted to (e.g., movable along) the length of the tibial-tendon support strap.

FIGS. 3 and 4 are isolated perspective views of opposite sides of the arch pad of FIGS. 1 and 2 in accordance with an exemplary embodiment.

FIG. 5 is a bottom perspective view of the PTTD-brace of FIG. 1, wherein FIG. 5 depicts an intermediate configuration of the tibial-tendon support strap during an exemplary method of wrapping the tibial-tendon support strap about an ankle-brace boot of the PTTD-brace in accordance with an exemplary PTTD-brace embodiment.

FIG. 6 is a side perspective view of the PTTD-brace of FIG. 1 with the tibial-tendon support strap encircling the ankle-brace boot of the PTTD-brace in accordance with an exemplary PTTD-brace embodiment.

FIG. 7 is a front view of the PTTD-brace of FIG. 1 with the tibial-tendon support strap encircling the ankle-brace boot of the PTTD-brace in accordance with an exemplary PTTD-brace embodiment.

FIG. 8 is like FIG. 7, except that a stabilizing strap is also encircling the PTTD-brace, in accordance with an exemplary PTTD-brace embodiment.

FIG. 9 is a bottom perspective view of the PTTD-brace embodiment of FIG. 8.

FIG. 10 is like FIG. 8, except that binding straps are also encircling the PTTD-brace, in accordance with an exemplary PTTD-brace embodiment.

FIGS. 11-14 are perspective views of an exemplary PTTD-brace embodiment having an advantageous lacing configuration, wherein the tibial-tendon support strap, and any stabilizing strap(s) and any support strap(s), are omitted.

### DETAILED DESCRIPTION

The present braces for posterior tibial tendon dysfunction (i.e., “PTTD”) are described herein with reference to the accompanying drawings, which depict exemplary embodiments that should not be construed as limiting the scope of the invention. Rather, the exemplary PTTD-brace embodiments are provided so this disclosure will be thorough and complete to convey to those having ordinary skill in the art the scope of the invention. In the accompanying drawings, like numbers refer to like elements.

As described herein, the terms “interior surface” and “inner surface” refer to a two-dimensional surface or side closest to the patient's ankle, and the terms “exterior surface” and “outer surface” refer to a two-dimensional surface or side farthest from the patient's ankle (i.e., an outer surface is opposite an inner surface).

The term “section” refers in context to a portion or an area of an ankle-support sleeve (e.g., a PTTD-brace sleeve). The term “proximal” refers to the part of the arch-support brace or lower leg that is closer to the patient's knee, and the term “distal” refers to the part of the arch-support brace or lower leg farther from the patient's knee.

The terms “positioned” and “positioning” are used conventionally to refer to one element being located relative to another element. This embraces one element being fixed or releasably secured to another element. For example, a first element may be positioned against a second element (or positioned between second and third elements) by sewing, by hook-and-loop fasteners, or by other known mechanisms for physically attaching physical elements to each other. The terms “fixed,” “affixed,” and “secured” may include sewn, made integral with, adhered with adhesive, bonded (e.g., fused with heat), or otherwise attached in any suitable manner.

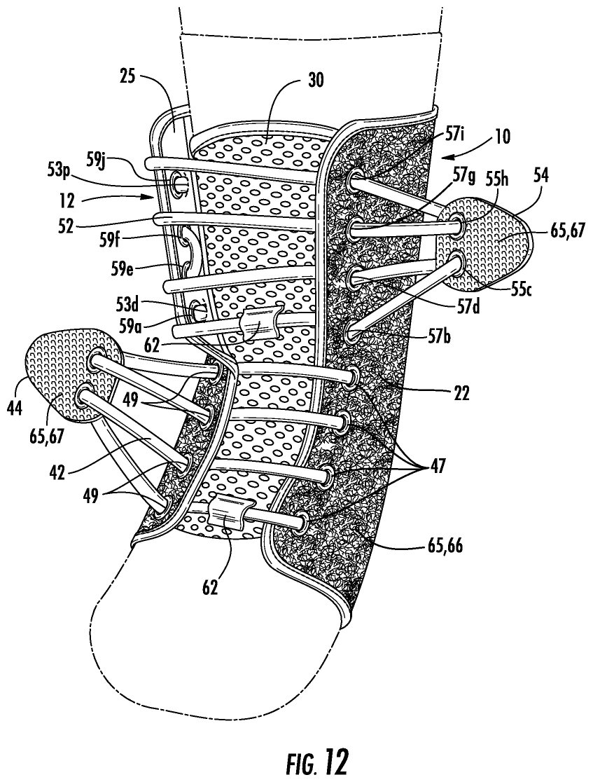

The terms “freely threaded” and “freely interlaced” are used herein in accordance with the PTTD-brace embodiments depicted in FIGS. 11-14 to refer to a lace that is moveably connected to a lace-redirection mechanism, such as an eyelet, rather than fixedly attached to a lace-redirection mechanism. For example, as described herein, a lace that is “freely threaded” through a closure-tab lace-redirection mechanism, which is positioned on or otherwise formed through a lacing closure tab, can facilitate movement of the lacing closure tab along the lace.

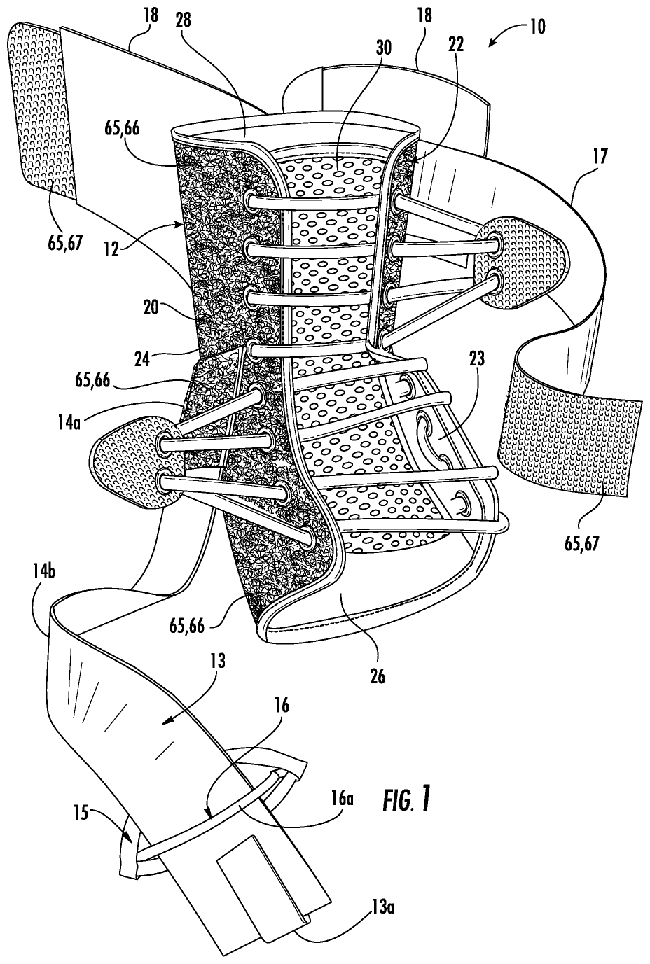

FIGS. 1-14 depict exemplary PTTD-brace embodiments, which are typically sized for a patient. These exemplary arch-support braces, which can be worn with or without athletic socks (and within or without a shoe), are typically configured to fit either a left foot or a right foot (i.e., typically an arch-support brace for a left foot is configured differently than an arch-support brace for a right foot). FIGS. 1-10 depict a right-foot configuration of an exemplary PTTD-brace.

As noted, this U.S. nonprovisional application incorporates by reference priority U.S. Patent Application No. 62/531,476 and priority U.S. Patent Application No. 62/596,388, including Appendix I, Appendix II, and Appendix III. Appendix I and Appendix II include photographs of an exemplary prototype of the right-foot arch-support brace depicted in FIGS. 1-10. (The photographs in Appendix II include indicator tape to better show the configuration of the respective straps as positioned on a foot and ankle.) Appendix III includes photographs of the same kind of exemplary prototype depicted in FIGS. 1-10, albeit for a left-foot configuration.

With reference to the exemplary arch-support brace 10 depicted in FIG. 1, the arch-support brace 10 includes an ankle-brace boot 12 defining an interior surface and an exterior surface. The ankle-brace boot 12, which is configured to receive a foot and an ankle, is partly formed by an ankle support sleeve 20, which includes at least a first side-sleeve section 22 and a second side-sleeve section 24. The first side-sleeve section 22 and the second side-sleeve section 24 represent opposite sides of the arch-support brace 10. For example, if the first side-sleeve section 22 is a left quarter panel of the ankle-brace boot 12, the second side-sleeve section 24 is a right quarter panel of the ankle-brace boot 12, and vice-versa. The first side-sleeve section 22 and the second side-sleeve section 24 typically define respective free front edges toward the front of the ankle support sleeve 20.

As herein described with respect to FIGS. 1-10, the first side-sleeve section 22 defines an inner section of the arch-support brace 10 corresponding to the patient's medial malleolus (i.e., the inner bony prominence of the ankle), and the second side-sleeve section 24 defines an outer section of the arch-support brace 10 corresponding to the patient's lateral malleolus (i.e., the outer bony prominence of the ankle). Accordingly, in the right-foot configuration of the arch-support brace as depicted in FIGS. 1-10, the first side-sleeve section 22 is an inside, left quarter panel of the ankle-brace boot 12, and the second side-sleeve section 24 is an outside, right quarter panel of the ankle-brace boot 12.

Referring to FIG. 1, the ankle support sleeve 20 may include an optional bottom-sleeve section 26 positioned beneath and between, and connected to, the first side-sleeve section 22 and the second side-sleeve section 24. When the arch-support brace 10 is secured to a patient's foot and ankle, the patient's rear sole should be placed against the bottom-sleeve section 26. Similarly, and with continued reference to FIG. 1, the ankle support sleeve 20 may include an optional rear-sleeve section 28 positioned between and connected to both the first side-sleeve section 22 and the second side-sleeve section 24. When the arch-support brace 10 is secured to a patient's foot and ankle, the patient's lower leg and Achilles tendon should be placed against the rear-sleeve section 28. The rear-sleeve section 28 may include cushioning to provide the patient with enhanced comfort and support. More generally, padding may be applied to the interior surfaces of the ankle-brace boot 12 to help engage the patient's ankle and to improve patient comfort.

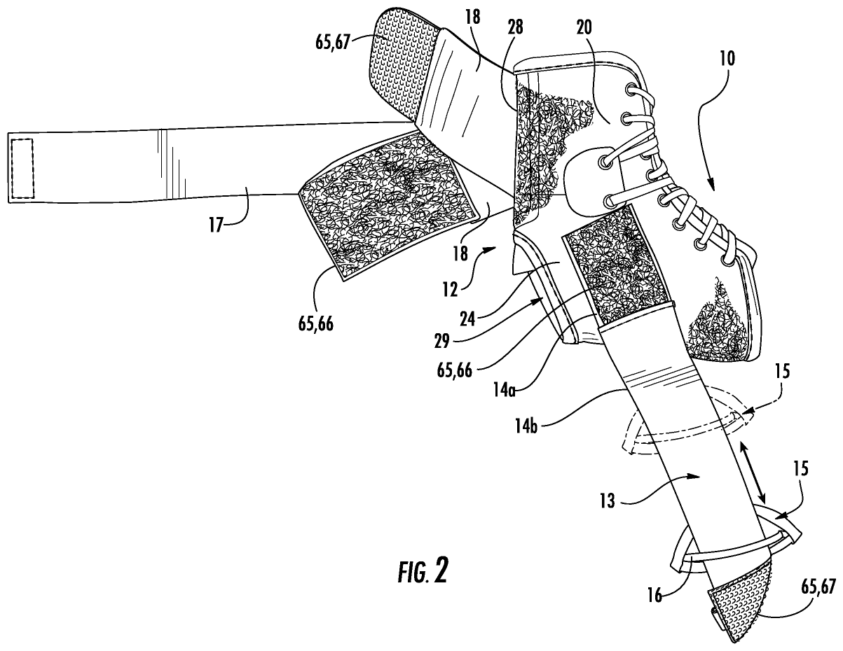

In exemplary PTTD-brace embodiments, the ankle support sleeve 20 is a boot-like body member that is substantially L-shaped to cover at least a lower and rear portion of the patient's foot and ankle. In these exemplary PTTD-brace embodiments, the ankle support sleeve 20 includes a lower portion (e.g., the bottom-sleeve section 26) that conforms to the lower surfaces of the foot by extending under portions of the patient's calcaneus. In these exemplary PTTD-brace embodiments, an example of which is shown in FIG. 2, the ankle support sleeve 20 typically defines a heel opening 29 (e.g., for receiving a portion of the patient's heel).

In other exemplary PTTD-brace embodiments, the ankle support sleeve 20 excludes a lower portion (e.g., the bottom-sleeve section 26) such that the ankle support sleeve 20 has a lower edge ending approximately below the malleoli (e.g., immediately below the malleoli). In these exemplary PTTD-brace embodiments, the ankle support sleeve 20 does not extend under the patient's foot and so is more U-shaped or C-shaped (i.e., in a top view) than L-shaped (i.e., in a side view).

To alleviate the painful symptoms of posterior tibial tendon dysfunction—and perhaps posterior tibial tendinitis, too—the arch-support brace 10 includes at least one tibial-tendon support strap 13 having a free portion (e.g., a free end) and an attached or attachable portion (e.g., an attached end). The attached-end portion of the tibial-tendon support strap 13 is typically secured to the lower, outer portion of the ankle support sleeve 20. For example, and as best understood with reference to the example depicted in FIG. 2, the tibial-tendon support strap 13 may be secured to the outer, second side-sleeve section 24 (e.g., near the heel opening 29 in the ankle support sleeve 20). In this regard, the tibial-tendon support strap 13 may be either fixedly secured or releasably secured to the outer, second side-sleeve section 24. In embodiments in which the tibial-tendon support strap 13 is fixedly secured to the outer, second side-sleeve section 24, the fixed securing may be provided by any suitable fastening technique(s). For example, the tibial-tendon support strap 13 may be sewn to the outer, second side-sleeve section 24. In embodiments in which the tibial-tendon support strap 13 is releasably secured to the outer, second side-sleeve section 24, the releasable securing may be provided by any suitable releasable fastening technique(s). For example, the tibial-tendon support strap 13 may be releasably secured to the outer, second side-sleeve section 24 by way of one or more fasteners, such as hook-and-loop fasteners 65 (e.g., VELCRO-brand fasteners). As a more general example, the tibial-tendon support strap 13 may be configured to be releasably secured to the outer, second side-sleeve section 24 in any suitable manner.

The tibial-tendon support strap 13 may be configured so that, while extending straight from its attachment to the second side-sleeve section 24 (e.g., its attachment point at the second side-sleeve section 24), the tibial-tendon support strap 13 extends obliquely at a slight forward offset from vertical. For example, the tibial-tendon support strap 13 may extend at an angle of inclination relative to vertical from the patient's planted foot to facilitate positioning of the tibial-tendon support strap 13 under the patient's mid-foot arch (e.g., the patient's medial arch). The angle of inclination may be defined relative to a predetermined axis. The upper portion of the ankle support sleeve 20 may extend generally or substantially cylindrically around (e.g., is configured to extend generally or substantially cylindrically around) the predetermined axis. For example, the angle of inclination may be in a range of from about zero degrees to about thirty degrees, in a range of from about five degrees to about thirty degrees, or in a range of from about ten degrees to about thirty degrees, including all values and subranges therebetween for each of these ranges.

In the embodiments depicted in the drawings, the attachment point of the attached-end portion of the tibial-tendon support strap 13 roughly corresponds to (e.g., is positioned to be roughly superposed with) the patient's lateral malleolus (i.e., the outer bony prominence of the ankle). However, the attachment point of the attached-end portion of the tibial-tendon support strap 13 may be in any suitable position, and such suitable positions may typically be generally proximate the patient's lateral malleolus. For example, the attachment point of the attached-end portion of the tibial-tendon support strap 13 may be below the patient's lateral malleolus. During patient use, the opposite end portion of the tibial-tendon support strap 13 (i.e., originally the free-end portion of the tibial-tendon support strap 13) is typically attached to the attached-end portion of the tibial-tendon support strap 13 in a superposed configuration.

At least a portion of the tibial-tendon support strap 13 may be elastic. For example, the tibial-tendon support strap 13 may include an elastic section 14a (e.g., about one to three inches) secured to a relatively inelastic section 14b that is less stretchable and less elastic as compared with the elastic section 14a. If present, an end portion of the elastic section 14a may be the portion of the tibial-tendon support strap 13 that is secured to (e.g., sewn upon) the outer portion of the ankle support sleeve 20. The elastic section 14a of the tibial-tendon support strap 13 helps maintain tension in the tibial-tendon support strap 13 and pressure upon the patient's posterior tibial tendon when the arch-support brace 10 is secured to the patient's foot and ankle.

Referring to FIGS. 1-4, the arch-support brace 10 may include an arch pad 15, which may be positioned by the patient on the inner, underside of the patient's arch. The arch pad 15 may be moveably connected to the tibial-tendon support strap 13, such as via the tibial-tendon support strap 13 extending through at least one arch-pad channel 16 (e.g., a loop) of the arch pad 15 and/or in any other suitable manner. As schematically depicted in FIG. 2 (i.e., by a double-ended arrow and the arch pad 15 being depicted in both dashed and solid lines), the arch-pad channel 16 facilitates repositioning of the arch pad 15 along the length of the tibial-tendon support strap 13. Alternatively, the arch pad 15 and tibial-tendon support strap 13 may be cooperatively configured with respective fasteners so that the arch pad 15 is releasably attachable to the tibial-tendon support strap 13 at different positions along the length of the tibial-tendon support strap 13, as will be further discussed herein.

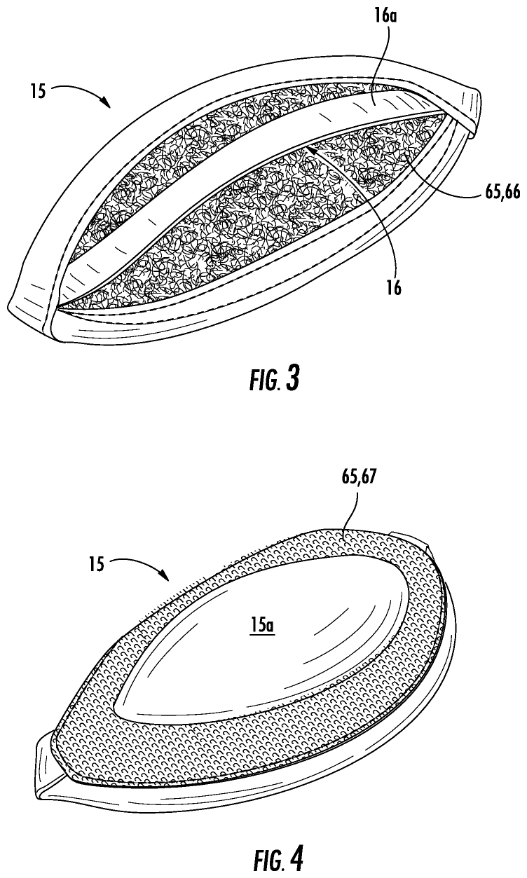

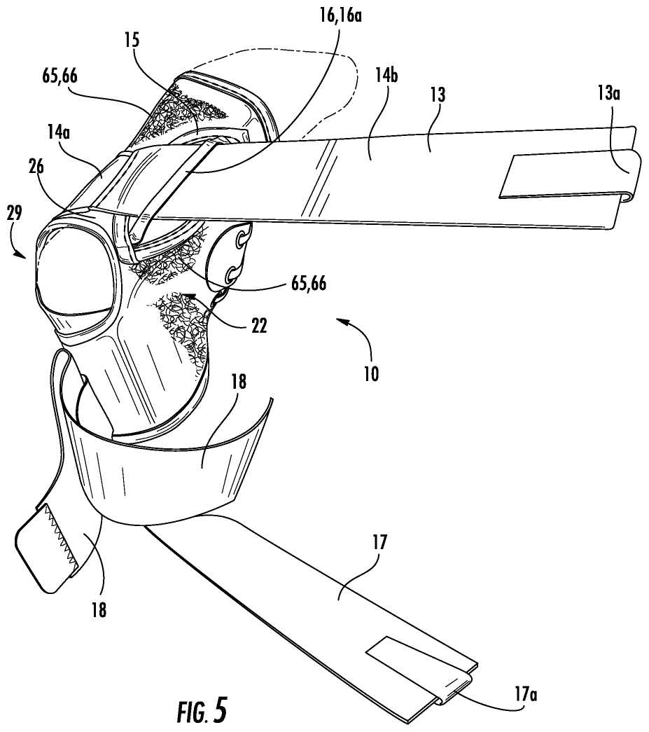

Referring to FIGS. 3 and 4, an exemplary arch pad 15 is contoured to correspond to the patient's medial arch (e.g., the patient's mid-foot arch). For example, the arch pad 15 can have, or at least partially have, a lentiform shape. The arch pad 15 may include layers of soft polymeric gel and memory foam, and reinforcing layers of firmer material (e.g., stiffening plastic). The arch pad 15 may include one or more fasteners, such as hook-and-loop fasteners 65 (e.g., VELCRO-brand fasteners), to releasably secure the arch pad 15 to one or more features of the arch-support brace 10. For example, the arch pad 15 may be releasably secured to the ankle support sleeve 20 (i.e., to the exterior of the bottom-sleeve section 26) at a position so that the arch pad 15 is superposed with the patient's mid-foot arch. In an alternative embodiment, an arch pad (not shown) is fixed to (e.g., sewn directly to) the tibial-tendon support strap 13. In another alternative embodiment, an arch pad (not shown) is fixed to (e.g., sewn directly to) the ankle support sleeve 20 and no repositioning of the arch pad is required. In yet another alternative embodiment, the arch-pad channel 16 may be omitted and the resulting arch pad (not shown) may be releasably secured to the ankle support sleeve 20 independently of (e.g., at least initially independently of) the tibial-tendon support strap 13.

In the embodiment of the arch pad 15 depicted in FIGS. 3 and 4, each of the opposite exterior surfaces of the arch pad 15 can include fasteners, such as hook-and-loop fasteners 65 (e.g., VELCRO-brand fasteners). Referring to FIG. 3, the depicted exterior surface of the arch pad 15 may include a surficial fabric that functions as a loop fastener, or loop-fasteners 66 (e.g., loop-fastener strip(s)) may be secured to the exterior surface of the arch pad 15. The arch-pad channel 16 may be partially defined by at least one strip of material 16a having opposite ends respectively fixed to the opposite end sections of the arch pad 15. More generally, the arch-pad channel 16, when present, may be formed in any other suitable manner.

In the embodiments depicted in the drawings (e.g., FIG. 4), fasteners, such as hook-and-loop fasteners 65 (e.g., VELCRO-brand fasteners), may releasably secure the arch pad 15 to the patient's foot. For example, the exterior surface or face of the arch pad 15 depicted in FIG. 4 typically includes hook-fasteners 67, which releasably engage loop-fasteners 66 of the ankle support sleeve 20 (FIG. 1). Referring to FIG. 1, the ankle support sleeve 20, including the exterior surface of the bottom-sleeve section 26, may include a surficial fabric that functions as a loop fastener, or loop-fasteners 66 (e.g., loop-fastener strip(s)) can be secured to the outer surfaces of the ankle support sleeve 20 (e.g., the ankle support sleeve's bottom-sleeve section 26).

In the embodiment depicted in FIG. 4, the collective arrangement of hook-fasteners 67 of the arch pad 15 extends at least partially around, or completely around, an outwardly bulging central surface area 15a of the arch pad. The exterior surface of the outwardly bulging central surface area 15a may protrude outwardly relative to, and be relatively smooth (e.g., have a lower coefficient of friction) as compared with, the hook-fasteners 67 that encircle the central surface area 15a. In this way, the hook-fasteners 67 that encircle the central surface area 15a may not become securely fastened to the respective loop-fasteners 66 of the ankle support sleeve 20 until the arch pad 15 is securely forced against the ankle support sleeve's bottom-sleeve section 26, such as by the tensioned tibial-tendon support strap 13.

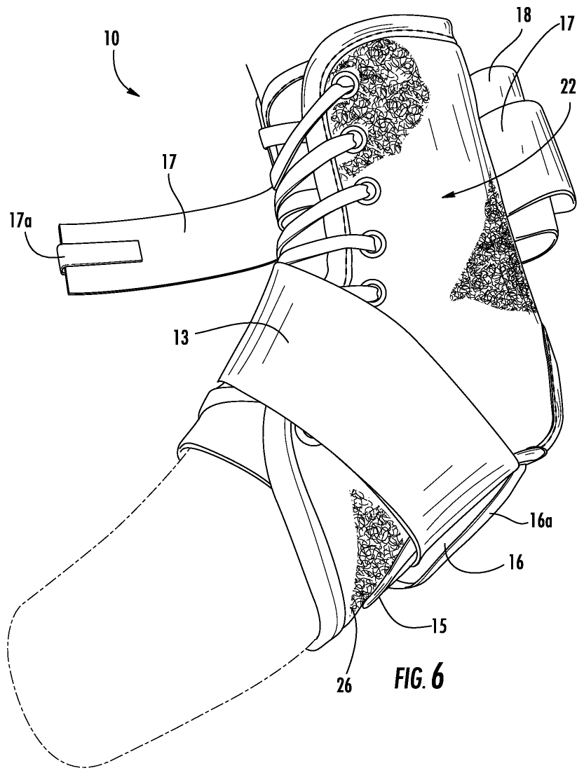

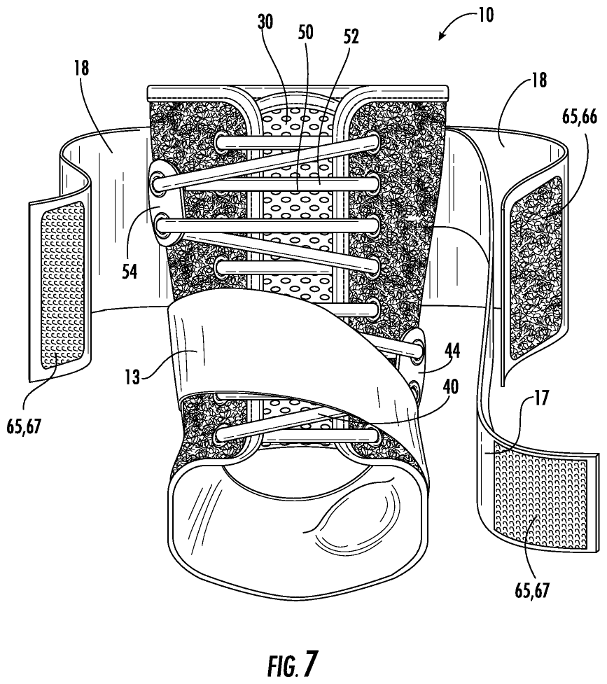

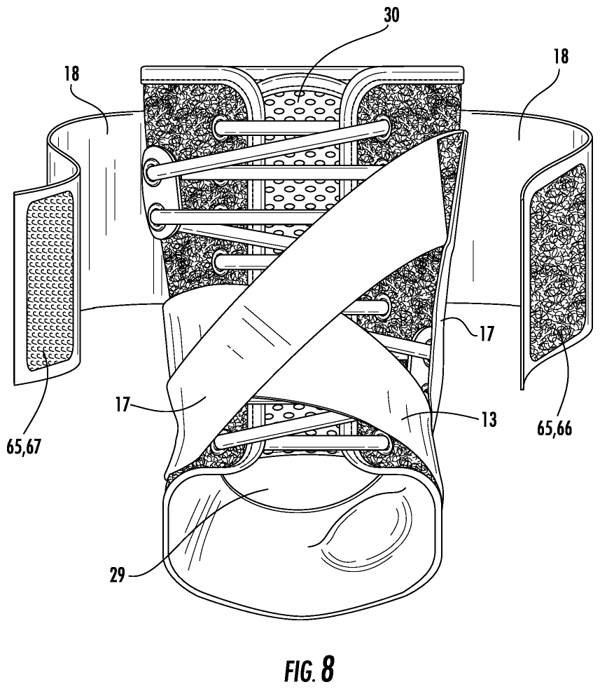

In accordance with an exemplary embodiment depicted in FIG. 5, the arch pad 15 is positioned on the inner, underside of the patient's medial arch; the tibial-tendon support strap 13 extends through the arch-pad channel 16; and the tibial-tendon support strap 13 is in an intermediate stage of being tightly wrapped around the patient's foot. FIGS. 6 and 7 depict an exemplary embodiment in which the tibial-tendon support strap 13 is tightly wrapped around the patient's foot, forcing the arch pad 15 against the ankle support sleeve's bottom-sleeve section 26. As depicted in FIGS. 5 and 6, the tibial-tendon support strap 13 traverses, and the arch pad 15 indirectly engages, the highest portion of the patient's medial arch. As such, a force is applied at least to the underside of the patient's medial arch.

When the arch-support brace 10 is secured to the patient's foot and ankle, the tibial-tendon support strap 13 passes from its attachment on the ankle support sleeve 20 (e.g., near the patient's lateral malleolus, such as shown in FIG. 2) underneath the patient's foot (e.g., near the talus and anterior calcaneus, such as shown in FIG. 5), and over the dorsum of the patient's foot (e.g., the anterior or upper foot surface, such as shown in FIGS. 6-7). As depicted in FIGS. 5-7, the tibial-tendon support strap 13 thus extends at least partially around, or more specifically encircles, the patient's foot via an under-and-over wrapping.

Without being bound to any theory, the tibial-tendon support strap 13 applies rotational and vertical forces to the patient's foot and ankle (i) to encourage inward heel rotation toward the opposite foot to help restore calcaneus alignment and (ii) to encourage combined lift and rotation for the mid-foot region to elevate the patient's arch. An exemplary tibial-tendon support strap 13 may be configured so that the tension in the tibial-tendon support strap 13 can be described as, or described by, a helical vector and/or a vector function that defines a helix (e.g., a vector function that approximately and/or substantially defines a helix or the like). Further regarding this aspect, the tibial-tendon support strap 13 may be configured (e.g., the attachment point of the attached-end portion of the tibial-tendon support strap 13 can be positioned) such that the tension in the tibial-tendon support strap 13 creates a predetermined, advantageous torsional effect on the rear and mid-foot sections of the patient's foot and ankle.

Referring to FIGS. 1 and 2, fasteners, such as hook-and-loop fasteners 65 (e.g., VELCRO-brand fasteners), releasably secure the tibial-tendon support strap 13 to the patient's foot. For example, the free-end portion of the tibial-tendon support strap 13 typically includes hook-fasteners 67, which releasably engage loop-fasteners 66. The ankle support sleeve 20, including the outer, second side-sleeve section 24, may include a surficial fabric that functions as a loop-fastener, or loop-fasteners 66 (e.g., loop-fastener strip(s)) can be secured to the outer surfaces of the ankle support sleeve 20 (e.g., the ankle support sleeve's second side-sleeve section 24). More typically, as further depicted in FIG. 9, the outer surface of the tibial-tendon-strap elastic section 14a includes loop-fasteners 66 so the tibial-tendon support strap 13 can encircle the patient's foot (i.e., under and over) before securing its free-end portion to its attached-end portion.

The free-end portion of the tibial-tendon support strap 13 is typically manually pulled so that the tibial-tendon support strap 13 is under tension while the free-end portion of the tibial-tendon support strap 13 is being secured. In this way, the tibial-tendon support strap 13 remains under tension after the free-end portion of the tibial-tendon support strap 13 is secured. For at least partially facilitating the pulling on the free-end portion of the tibial-tendon support strap 13, the free-end portion of the tibial-tendon support strap 13 may include at least one channel or loop 13a for receiving one or more fingers. The optional tibial-tendon support strap loop 13a may be at least partially defined by a strip of material having opposite ends fixed to the free-end portion of the tibial-tendon support strap 13, or the channel or loop 13a may be formed in any other suitable manner, or it may be omitted.

As noted, in some embodiments of the arch-support brace 10, the ankle support sleeve 20 excludes a lower portion (e.g., the bottom-sleeve section 26) such that the ankle support sleeve 20 has a lower edge ending approximately below the malleoli. In these exemplary PTTD-brace embodiments, the ankle support sleeve 20 does not extend under the patient's foot and so is U-shaped or C-shaped (i.e., in a top view) rather than L-shaped (i.e., in a side view). In such embodiments, the arch pad 15 may include one or more fasteners, such as hook-and-loop fasteners 65 (e.g., VELCRO brand fasteners), configured to releasably secure the arch pad 15 to corresponding fasteners of the tibial-tendon support strap 13 at a position so that the arch pad 15 is superposed with the patient's mid-foot arch (e.g., the patient's medial arch). For example, and as noted (above), the arch pad 15 and tibial-tendon support strap 13 may be cooperatively configured with respective fasteners so that the arch pad 15 is releasably attachable to the tibial-tendon support strap 13 at different positions along the length of the tibial-tendon support strap 13. In such embodiments, the tibial-tendon support strap 13 may include one or more fasteners, such as hook-and-loop fasteners 65 (e.g., VELCRO brand fasteners), positioned along the length of the tibial-tendon support strap 13 for attaching to corresponding fasteners of the arch pad 15. As noted, the arch-pad channel(s) 16 may be included or omitted.

Referring to FIGS. 2, 5, 6, and 7, the angle of inclination of the tibial-tendon support strap 13 relative to vertical from the patient's planted foot and/or the position of the attachment of the attached-end portion of the tibial-tendon support strap 13 can be configured (e.g., cooperatively configured) in a manner that allows the fully and securely installed tibial-tendon support strap 13 to advantageously lay flat (e.g., remain unpuckered and unpleated, or the like) while the tibial-tendon support strap 13 extends at least partially around the patient's foot (e.g., via an under-and-over wrapping) to apply a force to assist the patient's tibial tendon. As a more specific example, the angle of inclination of the tibial-tendon support strap 13 and/or the position of the attachment of the attached-end portion of the tibial-tendon support strap 13 can be configured (e.g., cooperatively configured) in a manner that establishes tension in the long direction of the tibial-tendon support strap 13 by equilibrating the compression (e.g., localized compression) across the strap's width. This allows the fully and securely installed tibial-tendon support strap 13 to advantageously lay flat (e.g., remain unpuckered and unpleated, or the like) while the tibial-tendon support strap 13 traverses, and the arch pad 15 engages, the highest portion of the patient's medial arch.

The arch-support brace 10 may optionally include at least one stabilizing strap 17 (e.g., an inner stabilizing strap) having a free portion (e.g., a free end) and an attached portion (e.g., a fixed end). The attached-end portion of the stabilizing strap 17 is typically secured to an upper portion of the ankle support sleeve 20. For example, the attached-end portion of the stabilizing strap 17 may be secured at or toward the rear of the ankle support sleeve 20, such as affixed to the rear-sleeve section 28 that is positioned between and connected to both the first side-sleeve section 22 and the second side-sleeve section 24. In other PTTD-brace embodiments, the stabilizing strap 17 may be secured to the first side-sleeve section 22, the second side-sleeve section 24, or both sections. The angle of inclination of the stabilizing strap 17 relative to vertical from the patient's planted foot and/or the position of the attachment of the attached-end portion of the stabilizing strap 17 (e.g., the attached portion of the stabilizing strap 17) can be configured (e.g., cooperatively configured) in a manner that establishes tension in the long direction of the stabilizing strap 17 by equilibrating the compression (e.g., localized compression) across the strap's width. This allows the fully and securely installed stabilizing strap 17 to advantageously lay flat (e.g., remain unpuckered and unpleated, or the like) while the stabilizing strap 17 extends at least partially around the patient's foot (e.g., via an over-and-under wrapping) to apply a force to the patient's heel (e.g., the anterior calcaneus).

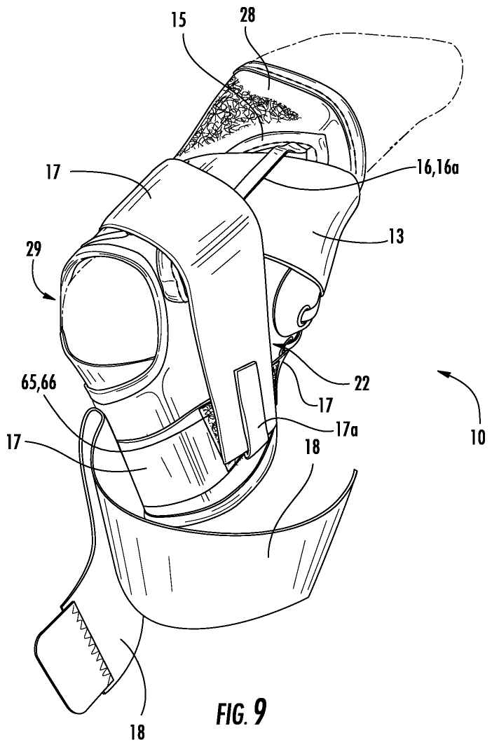

Referring to FIGS. 8 and 9, when the arch-support brace 10 is secured to the patient's foot and ankle, the stabilizing strap 17 passes from its fixation at the upper portion of the ankle support sleeve 20 over the dorsum of the patient's foot (i.e., the anterior or upper foot surface) and underneath the patient's heel area (e.g., near the anterior calcaneus). In the embodiment depicted in FIGS. 8 and 9, the stabilizing strap 17 crosses over the tibial-tendon support strap 13 and encircles the patient's foot and ankle in an over-and-under wrapping. The free-end portion of the stabilizing strap 17 extends upwardly from the patient's heel area along the exterior of the ankle support sleeve 20. In effect, an encircling configuration of the inner stabilizing strap 17 overlies the tibial-tendon support strap 13 while forming a heel stirrup. In an exemplary embodiment, the outer tibial-tendon support strap 13 is positioned underneath the patient's mid-foot arch (e.g., the patient's medial arch), and the inner stabilizing strap 17 is positioned underneath the patient's heel area (e.g., near the anterior calcaneus) to provide an advantageous torsional effect on the patient's foot and ankle. Fasteners, such as hook-and-loop fasteners 65 (e.g., VELCRO-brand fasteners), releasably secure the free-end portion of the stabilizing strap 17 to the ankle support sleeve 20 (e.g., to the inner, first side-sleeve section 22) and/or to the attached-end portion of the stabilizing strap 17.

The free-end portion of the stabilizing strap 17 is typically manually pulled so that the stabilizing strap 17 is under tension while the free-end portion of the stabilizing strap 17 is being secured. In this way, the stabilizing strap 17 remains under tension after the free-end portion of the stabilizing strap 17 is secured. For at least partially facilitating the pulling on the free-end portion of the stabilizing strap 17, the free-end portion of the stabilizing strap 17 may include at least one channel or loop 17a for receiving one or more fingers. The stabilizing strap loop 17a may be at least partially defined by a strip of material having opposite ends fixed to the free-end portion of the stabilizing strap 17, or the channel or loop 17a may be formed in any other suitable manner, or it may be omitted.

Without being bound to any theory, the optional stabilizing strap 17 supplements the tibial-tendon support strap 13 in encouraging combined lift and rotation of the mid-foot region to elevate the patient's arch. Referring to FIG. 9, when the arch-support brace 10 is secured to the patient's foot and ankle, the tibial-tendon support strap 13 and the stabilizing strap 17 create a “V-shape” or oblique arrangement of overlapping straps near the arch pad 15, which, as noted, is typically positioned on the inner, underside of the patient's arch (e.g., the patient's medial arch).

The embodiments of the arch-support brace 10 depicted in the drawings show a single stabilizing strap 17, which may be typical and/or advantageous in some situations. The present disclosure, however, is not limited to the inclusion of only a single stabilizing strap 17. A PTTD-brace embodiment might optionally include at least two stabilizing straps 17. For example, the respective stabilizing straps 17 may encircle the patient's foot in the same direction or the opposite direction and be releasably secured respectively to the same side or the opposite side of the ankle support sleeve 20 (e.g., to the inner, first side-sleeve section 22 and/or to the outer, second side-sleeve section 24). That said, such a configuration may not be typical.

The arch-support brace 10 may optionally include one or more binding straps 18. The binding strap(s) 18 typically include elastic material to permit stretching. For example, two binding straps 18 may be provided by attaching a middle portion of an elastic strip to the rear-sleeve section 28 of the ankle support sleeve 20. Alternatively, the binding strap(s) 18 may be provided in any other suitable manner. As depicted in FIGS. 1, 2, and 5-10, the arch-support brace 10 typically includes two binding straps 18, each having a free portion (e.g., a free end) and a fixed portion (e.g., a fixed end). The attached-end portion of each binding strap 18 is typically secured to an upper portion of the ankle support sleeve 20, such as affixed to the rear-sleeve section 28 that is positioned between and connected to both the first side-sleeve section 22 and the second side-sleeve section 24.

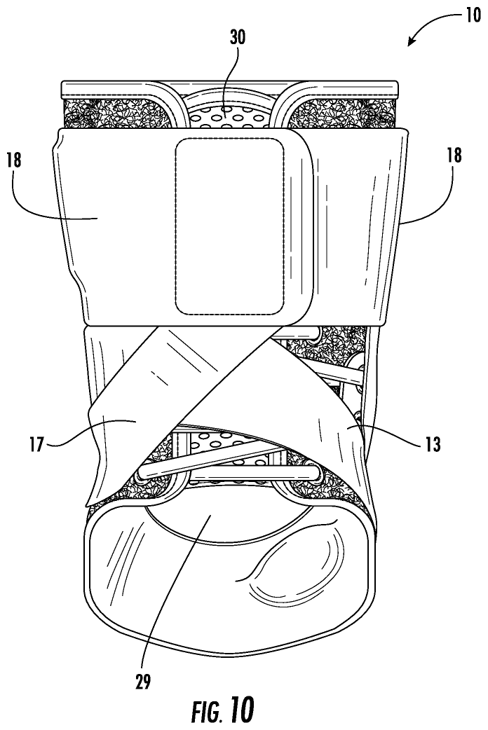

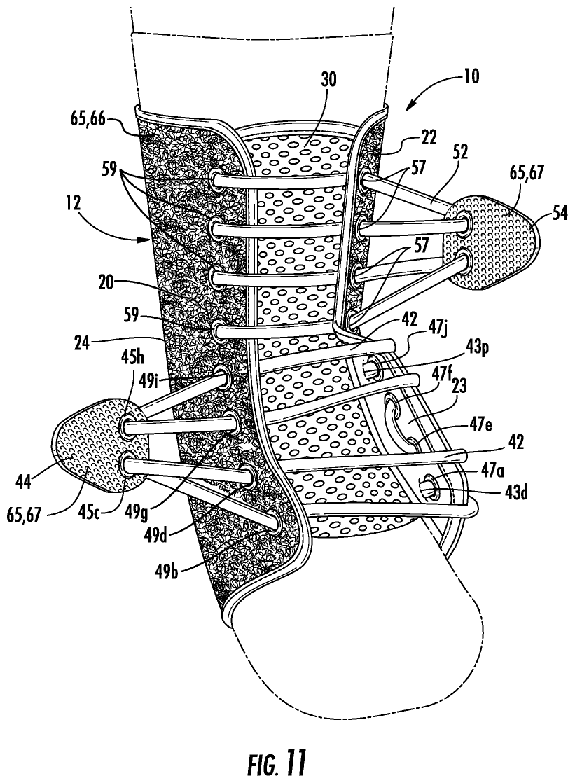

Fasteners, such as hook-and-loop fasteners 65 (e.g., VELCRO-brand fasteners), releasably secure the respective free ends of the binding straps 18 to the ankle support sleeve 20 or, more typically, to each other as shown in FIG. 10. In an exemplary embodiment, one of the binding straps includes loop-fasteners 66 (e.g., the outer surface of the first binding strap 18 has loop-fasteners 66), and the other binding strap includes hook-fasteners 67 (e.g., the inner surface of the second binding strap 18 has hook-fasteners 66) so that the respective binding straps overlie and engage one another. The respective binding straps 18 circumferentially wrap the patient's lower leg to further secure the arch-support brace 10 to the patient's foot and ankle.

Although FIGS. 1, 2, and 5-10 show two binding straps 18, an alternative embodiment employs a single binding strap 18 configured to wrap itself around the patient's lower leg. For example, a single binding strap 18 with an attached-end portion and a free-end portion may include fasteners, such as hook-and-loop fasteners 65, to releasably secure the binding strap 18 to the ankle support sleeve 20 or, more typically, to itself. The one or more binding straps 18 may each be more generally referred to as straps. Similarly, the tibial-tendon support strap 13 and the one or more stabilizing straps 17 may each be more generally referred to as straps.

Typically, the ankle support sleeve 20 is substantially inelastic (e.g., formed from flexible, low-stretch material) to help stabilize the patient's ankle and lower leg. In another exemplary embodiment, the rear-sleeve section 28 includes heel cushioning (e.g., via elastic material, such as stretchable, mesh fabric) positioned above the heel opening 29. The ankle support sleeve 20 may be constructed of substantially inelastic fabric material that resists stretching and elongation. In an exemplary PTTD-brace embodiment, the substantially inelastic material is lightweight nylon having excellent strength and durability (e.g., woven ballistic nylon fabric). As used herein, the term “elastic” generally refers to material that can be readily stretched or expanded, and then can return to its initial shape (i.e., elastic materials resist permanent deformation by stretching), and the term “inelastic” generally refers to material that resists stretching and elongation.

In another exemplary PTTD-brace embodiment, the ankle-brace boot 12 includes a tongue 30 (e.g., a front-panel tongue), which is typically connected to the ankle support sleeve's first side-sleeve section 22 and the ankle support sleeve's second side-sleeve section 24. The tongue 30, which is usually positioned between the first side-sleeve section's free front edge and the second side-sleeve section's free front edge, is typically affixed to the ankle support sleeve 20. For example, the tongue 30 may be secured (e.g., tacked or stitched) to the ankle support sleeve 20 only at or near the respective distal ends of the first side-sleeve section 22 and the second side-sleeve section 24. As depicted in FIG. 1, the tongue 30 may be positioned between and inwardly recessed relative to the first side-sleeve section's free front edge and the second side-sleeve section's free front edge.

Typically, the tongue 30 is stretchable to facilitate placement of the arch-support brace 10 on a patient's ankle and cushioned for the patient's comfort. For example, the tongue 30 may be formed of stretchable, mesh fabric. The tongue 30 facilitates placement of the ankle-brace boot 12 to the patient's foot and ankle and provides padding between the PTTD-brace laces and the patient's foot and ankle.

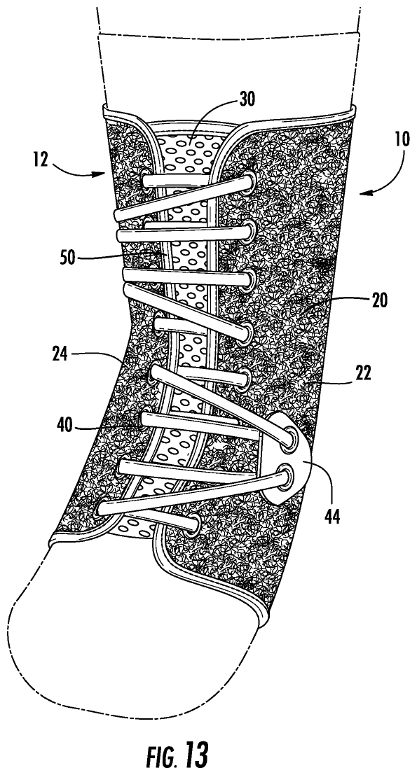

Together, the ankle support sleeve 20 and the tongue 30 substantially form the ankle-brace boot 12. For example, the tongue 30 closes the gap (e.g., the throat) in the ankle support sleeve 20, thereby forming the ankle-brace boot 12. The tongue 30, which may be secured to the ankle support sleeve 20 only at or near the respective distal ends of the first side-sleeve section 22 and the second side-sleeve section 24, is typically positioned within the gap or throat in the ankle support sleeve 20 so as to extend along both a first outer side-sleeve strip 23 (FIGS. 1 and 11) and second outer side-sleeve strip 25 (FIG. 12). The first outer side-sleeve strip 23, which is typically a marginal portion of the first side-sleeve section 22, may extend from proximate the tongue 30 to an adjacent outer edge of the first side-sleeve section 22. The second outer side-sleeve strip 25, which is typically a marginal portion of the second side-sleeve section 24, may extend from proximate the tongue 30 to an adjacent outer edge of the second side-sleeve section 24. For example, when the arch-support brace 10 is secured to the patient's foot and ankle, the first outer side-sleeve strip 23 may be partly bounded by the tongue 30 and an adjacent outer edge of the first side-sleeve section 22, and the second outer side-sleeve strip 25 may be partly bounded by the tongue 30 and an adjacent outer edge of the second side-sleeve section 24.

Eyelets (or similar holes or openings, such as reinforced holes and/or slots, or other lace-redirection mechanisms, such as loops, hooks, folded webbing, and/or buckles) are typically formed through or otherwise positioned at or near (e.g., directly or indirectly connected to or otherwise positioned upon) both the first side-sleeve section 22 and the second side-sleeve section 24. In an exemplary PTTD-brace embodiment, a first series of lace-redirection mechanisms are positioned at or near the ankle support sleeve's first side-sleeve section 22 (e.g., a first series of eyelets formed at a free front edge near the tongue 30), and a second series of lace-redirection mechanisms are positioned at or near the ankle support sleeve's second side-sleeve section 24 (e.g., a second series of eyelets formed at a free front edge near the tongue 30). One or more laces are then freely threaded (or otherwise freely interlaced) through these respective lace-redirection mechanisms (e.g., the laces are not fixedly attached to the respective lace-redirection mechanisms and can move through or along the lace-redirection mechanisms). For example, a single lace may be threaded (or otherwise interlaced) through eyelets formed on either side of the ankle support sleeve in a conventional manner or as disclosed in commonly assigned U.S. Pat. No. 8,808,215 and commonly assigned U.S. Pat. No. 9,375,339.

In this regard, the first outer side-sleeve strip 23 and the second outer side-sleeve strip 25 can function as eyestays for the arch-support brace 10. For example, first and second outer side-sleeve strips 23, 25 may be reinforced marginal portions of the first and second side-sleeve sections 22, 24, respectively. Those having ordinary skill in the art will understand that exemplary arch-support braces according to the present invention may employ any lace-redirection mechanisms (e.g., reinforced holes and slots, or external loops, hooks, folded webbing, and/or buckles) in addition to or instead of eyelets.

Moreover, it is within the scope of the present PTTD-brace invention to include one or more external flaps (not shown) on the exterior of the ankle support sleeve to facilitate redirection of a lace. In one PTTD-brace embodiment, a first flap having a series of eyelets or other lace-redirection mechanisms can be positioned upon the ankle support sleeve's first side-sleeve section (e.g., near the tongue). A lace may be guided through the first flap's lace-redirection mechanisms (e.g., eyelets) and a series of eyelets or other lace-redirection mechanisms positioned upon the ankle support sleeve's second side-sleeve section to promote even closure of the ankle-brace boot to optionally achieve cast-like compression on a patient's lower leg, ankle, and foot. In another PTTD-brace embodiment, a second flap having a series of eyelets or other lace-redirection mechanisms can be positioned upon the ankle support sleeve's second side-sleeve section (e.g., near the tongue). A lace may be guided through the second flap's lace-redirection mechanisms (e.g., eyelets) and a series of eyelets or other lace-redirection mechanisms positioned upon the ankle support sleeve's first side-sleeve section to promote even closure of the ankle-brace boot to optionally achieve cast-like compression on a patient's lower leg, ankle, and foot. In yet another PTTD-brace embodiment, a lace may be guided through both (i) the first flap having eyelets or other lace-redirection mechanisms and (ii) the second flap having eyelets or other lace-redirection mechanisms to optionally achieve cast-like compression on a patient's lower leg, ankle, and foot, thereby restricting flexion, extension, and lateral movement of the ankle joint. Such first and second flaps can indirectly connect the lace-redirection mechanisms to the respective first and second side-sleeve sections.

The ankle support sleeve 20 may be formed from one or more sheets of fabric. In one embodiment, the ankle support sleeve 20 is a “one-piece” body member formed from a single sheet of material that is capable of forming an L-shaped, boot-like shape (e.g., the ankle-brace boot 12 without the tongue 30). In another embodiment, the ankle support sleeve 20 is a “multi-piece” body member formed from two or more sheets or strips of material to form an L-shaped, boot-like shape (e.g., the ankle-brace boot 12 without the tongue 30). Similarly, “one-piece” and “multi-piece” body members in which the ankle support sleeves do not extend under the patient's foot are within the scope of the present disclosure.

The present arch-support brace for alleviating the painful symptoms of posterior tibial tendon dysfunction may include any suitable lacing configuration, or the like, such as an advantageous lacing configuration depicted in FIGS. 11-14, which omits the tibial-tendon support strap, the optional stabilizing strap, and the optional binding straps. The description of the abridged PTTD-brace embodiment depicted in FIGS. 11-14 typically applies to the PTTD-brace embodiment depicted in FIGS. 1-10, except as noted in the supporting description, figures, and photographs.

Exemplary arch-support braces may employ one or more lacing interconnection systems. Any suitable lacing interconnection system, or more generally any other suitable interconnection system, may be used. A typical lacing interconnection system according to the present disclosure includes (i) lace-redirection mechanisms positioned on the ankle-brace boot (e.g., respective series of eyelets positioned at the ankle support sleeve's first side-sleeve section and second side-sleeve section), (ii) a lacing closure tab (e.g., including one or more closure-tab eyelets or other lace-redirection mechanisms, such as reinforced holes and/or slots, or external loops, hooks, folded webbing, and/or buckles), and (iii) a lace attached to and connecting the ankle-brace boot and the lacing closure tab. When secured to the patient's ankle via the lacing interconnection system, an exemplary arch-support brace may optionally restrict flexion, extension, and lateral movement of the patient's ankle joint to help provide cast-like compression of the arch-support brace to a patient's lower leg, ankle, and foot.

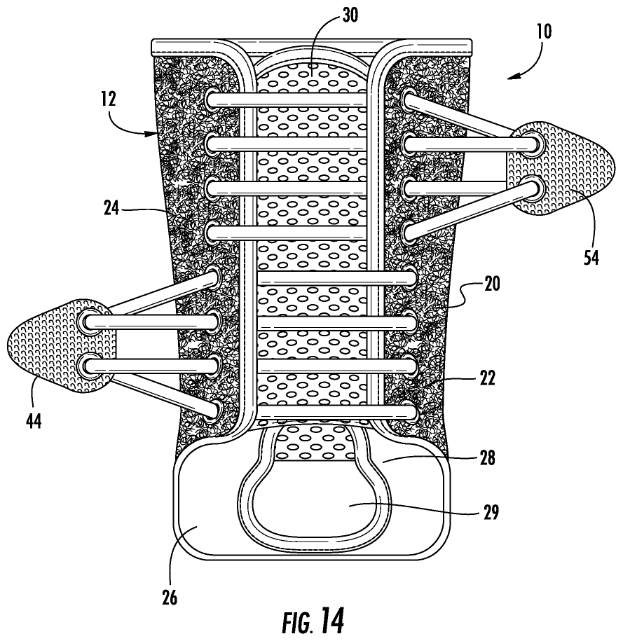

The exemplary arch-support brace embodiment depicted in FIGS. 11-14 includes two lacing interconnection systems, namely a lower lacing interconnection system 40, which is nearer the patient's toe when the arch-support brace is worn, and an upper lacing interconnection system 50, which is nearer the patient's knee when the arch-support brace is worn. As viewed from the patient's perspective and as depicted in FIG. 13, the lower lacing interconnection system 40 closes from right to left, and the upper lacing interconnection system 50 closes from left to right. As viewed from the patient's perspective (and to describe representative PTTD-brace embodiments), the ankle support sleeve's first side-sleeve section 22 is positioned to the left, and the ankle support sleeve's second side-sleeve section 24 is positioned to the right. Although such a reciprocal configuration of the lacing interconnection systems 40, 50 is typical, alternative PTTD-brace embodiments may employ parallel configurations, whereby both the lower lacing interconnection system 40 and the upper lacing interconnection system 50 close from right to left (or from left to right). Even though the exemplary arch-support braces are configured to fit either a left foot or a right foot (i.e., typically an arch-support brace for a left foot is configured differently than an arch-support brace for a right foot), the references to first and second sides (e.g., left and right from the patient's perspective for a right-foot configuration) are for purposes of description and not for limitation.

The exemplary lower lacing interconnection system 40 depicted in FIGS. 11-14 includes a lower lace 42 (e.g., exactly one lace) that moveably connects a lower lacing closure tab 44 (via one or more lace-redirection mechanisms, such as lower closure-tab eyelets 45) to lower lace-redirection mechanisms (e.g., lower eyelets 47, 49) positioned on the ankle support sleeve 20. Those having ordinary skill in the art will appreciate that the lower lacing closure tab 44 is moveably connected to the lower lace 42. When the lower lacing closure tab 44 is not releasably secured to another part of the arch-support brace (e.g., releasably affixed to the ankle support sleeve 20), the lower lacing closure tab 44 can move substantially freely along the lower lace 42 (e.g., the lower lace 42 can pass through the lower closure-tab eyelets 45).

With reference to the exemplary PTTD-brace embodiment depicted in FIGS. 11-14, four lower first side-sleeve eyelets 47 are formed through or otherwise positioned upon the ankle support sleeve's first side-sleeve section 22 near its free front edge (e.g., four lower eyelets are positioned in the first outer side-sleeve strip 23), and lower second side-sleeve eyelets 49 are formed through or otherwise positioned upon the ankle support sleeve's second side-sleeve section 24 near its free front edge (e.g., four lower eyelets are positioned in the second outer side-sleeve strip 25).

The lower lacing closure tab 44 includes a releasable-attachment mechanism, such as a hook-and-loop surface, to releasably secure the lower lacing closure tab 44 to another part of the arch-support brace 10. As illustrated in FIGS. 11-14, two lower closure-tab eyelets 45c, 45h are formed through or otherwise positioned upon the lower lacing closure tab 44. The lower closure-tab eyelets 45 help to ensure that the lower lacing closure tab 44 remains centered on the ankle support sleeve 20 as the lower lacing closure tab 44 is pulled over and around the ankle-brace boot 12.

The lower lace 42 is freely threaded (or otherwise interlaced) through the lower first side-sleeve eyelets 47, the lower second side-sleeve eyelets 49, and the lower closure-tab eyelets 45, respectively. The lower lace 42 can move freely to accommodate the typical non-cylindrical anatomy of a patient's lower leg in which the leg tapers from the calf to the ankle (e.g., a frustoconical shape). This free movement of the lower lace 42 facilitates consistent tension along its length. This even tension is transferred to the lower first side-sleeve eyelets 47, the lower second side-sleeve eyelets 49, and the lower closure-tab eyelets 45 through which the lower lace 42 is interlaced. The lower lace 42 and the moveably attached lower lacing closure tab 44 wrap circumferentially around—rather than helically along—the patient's lower leg and ankle in a way that uniformly closes and tensions the ankle-brace boot 12.

Typically, a lower lace 42 has (i) a first end portion secured (e.g., affixed) to the ankle support sleeve 20 near the ankle support sleeve's distal end (e.g., secured at either the distal end of the first outer side-sleeve strip 23 or the distal end of the second outer side-sleeve strip 25) and (ii) a second end portion secured (e.g., affixed) to the ankle support sleeve 20 approximately midway along the ankle support sleeve 20 (e.g., secured midway along the ankle support sleeve 20 toward the proximal end of the first outer side-sleeve strip 23 or the proximal end of the second outer side-sleeve strip 25). In the exemplary arch-support brace embodiment depicted in FIGS. 1-4, one lower lace 42 has (i) a first distal end fixed to the ankle support sleeve's interior surface nearer the ankle support sleeve's distal end (i.e., a lower distal fixation 43d toward the toe-side end of the first outer side-sleeve strip 23) and (ii) a second proximal end fixed to the ankle support sleeve's interior surface nearer the ankle support sleeve's proximal end (i.e., a lower proximal fixation 43p secured midway along the ankle support sleeve 20 toward the knee-side end of the first outer side-sleeve strip 23). In the exemplary PTTD-brace embodiment depicted in FIGS. 11-14, four lower first side-sleeve eyelets 47a, 47e, 47f, 47j are positioned at the ankle support sleeve's first side-sleeve section 22 between the lower lace's lower distal fixation 43d and the lower lace's lower proximal fixation 43p, and four lower second side-sleeve eyelets 49b, 49d, 49g, 49i are positioned at the ankle support sleeve's second side-sleeve section 24. As noted, the first outer side-sleeve strip 23 and the second outer side-sleeve strip 25 can function as eyestays (e.g., the four lower first side-sleeve eyelets 47a, 47e, 47f, 47j can be formed through the first outer side-sleeve strip 23, and the four lower second side-sleeve eyelets 49b, 49d, 49g, 49i can be formed through second outer side-sleeve strip 25).

As illustrated in FIGS. 11-14 in which the ends of lower lace 42 are fixed to the interior surface of the first side-sleeve section 22 (e.g., at the first outer side-sleeve strip 23 on the left side as viewed from the patient's perspective), pulling the lower lacing closure tab 44 away from the ankle support sleeve's second side-sleeve section 24 (and over and around the ankle support sleeve's first side-sleeve section 22) uniformly closes and tensions the ankle-brace boot 12 by drawing together the ankle support sleeve's second side-sleeve section 24 and ankle support sleeve's first side-sleeve section 22. As viewed from the patient's perspective, the lower lacing interconnection system 40 closes from right to left as shown in the exemplary arch-support brace of FIGS. 11-14.

As illustrated in the exemplary PTTD-brace embodiment depicted in FIGS. 11-14, the arch-support brace 10 includes a compression-enhancing lacing configuration that readily equilibrates (e.g., simultaneously evens) the tension in the lower lace 42 as the lower lacing closure tab 44 is extended from the ankle-brace boot 12. From the distal end of the arch-support brace 10, the lower lace 42 passes from its lower distal fixation 43d at the interior surface of the ankle support sleeve's first side-sleeve section 22 to and through an optional lower first outer distal eyelet 47a as depicted in FIG. 1.

With respect to FIGS. 11-14 as viewed from the patient's perspective, the ankle support sleeve's first side-sleeve section 22 is to the patient's left and the ankle support sleeve's second side-sleeve section 24 is to the patient's right. In one alternative embodiment (not shown), the distal end portion of the lower lace 42 can be fixed to the tongue 30 or another part of the ankle-brace boot 12 (e.g., an eyelet). In another alternative embodiment (not shown), the lower lace 42 is not immovably affixed to the ankle support sleeve 20 but rather knotted or otherwise configured (e.g., enlarged) so that the distal end portion of the lower lace 42 cannot pass through the lower first outer distal eyelet 47a or the like. For example, an end portion of the lower lace 42 can be doubled over, looped, or otherwise provided with any other suitable kind of obstruction, such as an enlargement (e.g., formed by deforming or melting), a whipping knot, a T-bar, or the like. In yet another alternative embodiment (not shown), the lower lace 42 can be fixed (e.g., stitched) to the exterior surface of the ankle support sleeve's first side-sleeve section 22, thereby rendering unnecessary the lower first outer distal eyelet 47a.

As illustrated in the exemplary ankle-brace embodiment depicted in FIG. 11, from the lower first outer distal eyelet 47a, the lower lace 42 passes to and through a lower second outer distal eyelet 49b, to and through a lower distal closure-tab eyelet 45c, to and through a lower second inner distal eyelet 49d, and to and through a lower first inner distal eyelet 47e. The lower lace 42 then passes lengthwise along the interior surface of the ankle support sleeve's first side-sleeve section 22 (e.g., along the interior surface of the first outer side-sleeve strip 23) from the lower first inner distal eyelet 47e to and through a lower first inner proximal eyelet 47f so that, between the lower first inner distal eyelet 47e and the lower first inner proximal eyelet 47f, the lower lace 42 is substantially parallel to the adjacent outer edge of the ankle support sleeve 20. The lower lace 42 then passes from the lower first inner proximal eyelet 47f to and through a lower second inner proximal eyelet 49g, to and through a lower proximal closure-tab eyelet 45h, to and through a lower second outer proximal eyelet 49i, to and through an optional lower first outer proximal eyelet 47j, and to a lower proximal fixation 43p at the interior surface of the ankle support sleeve's first side-sleeve section 22.

In one alternative embodiment (not shown), the proximal end portion of the lower lace 42 can be fixed to the tongue 30 or another part of the ankle-brace boot 12 (e.g., an eyelet). In another alternative embodiment (not shown), the lower lace 42 is not immovably affixed to the ankle support sleeve 20 but rather knotted or otherwise configured (e.g., enlarged) so that the proximal end portion of the lower lace 42 cannot pass through the lower first outer proximal eyelet 47j or the like. For example, an end portion of the lower lace 42 can be doubled over, looped, or otherwise provided with any other suitable kind of obstruction, such as an enlargement (e.g., formed by deforming or melting), a whipping knot, a T-bar, or the like. In yet another alternative embodiment (not shown), the lower lace 42 can be fixed (e.g., stitched) to the exterior surface of the ankle support sleeve's first side-sleeve section 22, thereby rendering unnecessary the lower first outer proximal eyelet 47j.

The exemplary arch-support brace embodiment depicted in FIGS. 11-14 also includes a reciprocal, upper lacing interconnection system 50. Whereas the lower lacing interconnection system 40 closes from right to left from the patient's perspective, the upper lacing interconnection system 50 closes from left to right.

The exemplary upper lacing interconnection system 50 shown in FIGS. 11-14 includes an upper lace 52 (e.g., exactly one lace) that moveably connects an upper lacing closure tab 54 (via one or more lace-redirection mechanisms, such as upper closure-tab eyelets 55) to upper lace-redirection mechanisms (e.g., upper eyelets 57, 59) positioned on the ankle support sleeve 20. Those having ordinary skill in the art will appreciate that the upper lacing closure tab 54 is moveably connected to the upper lace 52. When the upper lacing closure tab 54 is not releasably secured to another part of the arch-support brace (e.g., releasably affixed to the ankle support sleeve 20), the upper lacing closure tab 54 can move substantially freely along the upper lace 52 (e.g., the upper lace 52 can pass through the upper closure-tab eyelets 55).

With reference to the exemplary PTTD-brace embodiment depicted in FIGS. 11-14, four upper first side-sleeve eyelets 57 are formed through or otherwise positioned upon the ankle support sleeve's first side-sleeve section 22 near its free front edge (e.g., four upper eyelets are positioned in the first outer side-sleeve strip 23), and upper second side-sleeve eyelets 59 are formed through or otherwise positioned upon the ankle support sleeve's second side-sleeve section 24 near its free front edge (e.g., four upper eyelets are positioned in the second outer side-sleeve strip 25).

The upper lacing closure tab 54 includes a releasable-attachment mechanism, such as a hook-and-loop surface, to releasably secure the upper lacing closure tab 54 to another part of the arch-support brace 10. As illustrated in FIGS. 11-14, two upper closure-tab eyelets 55c, 55h are formed through or otherwise positioned upon the upper lacing closure tab 54. The upper closure-tab eyelets 55 help to ensure that the upper lacing closure tab 54 remains centered on the ankle support sleeve 20 as the upper lacing closure tab 54 is pulled over and around the ankle-brace boot 12.

The upper lace 52 is freely threaded (or otherwise interlaced) through the upper first side-sleeve eyelets 57, the upper second side-sleeve eyelets 59, and the upper closure-tab eyelets 55, respectively. The upper lace 52 can move freely to accommodate the typical non-cylindrical anatomy of a patient's lower leg in which the leg tapers from the calf to the ankle (e.g., a frustoconical shape). This free movement of the upper lace 52 facilitates consistent tension along its length. This even tension is transferred to the upper first side-sleeve eyelets 57, the upper second side-sleeve eyelets 59, and the upper closure-tab eyelets 55 through which the upper lace 52 is interlaced. The upper lace 52 and the moveably attached upper lacing closure tab 54 wrap circumferentially around—rather than helically along—the patient's lower leg in a way that uniformly closes and tensions the ankle-brace boot 12.

Typically, an upper lace 52 has (i) a first end portion secured (e.g., affixed) to the ankle support sleeve 20 approximately midway along the ankle support sleeve 20 (e.g., secured midway along the ankle support sleeve 20 toward the distal end of the first outer side-sleeve strip 23 or the distal end of the second outer side-sleeve strip 25) and (ii) a second end portion secured (e.g., affixed) to the ankle support sleeve 20 near the ankle support sleeve's proximal end (e.g., secured at either the proximal end of the first outer side-sleeve strip 23 or the proximal end of the second outer side-sleeve strip 25).

In the exemplary arch-support brace embodiment depicted in FIGS. 1-4, one upper lace 52 has (i) a first distal end fixed to the ankle support sleeve's interior surface nearer the ankle support sleeve's distal end (i.e., an upper distal fixation 53d secured midway along the ankle support sleeve 20 toward the toe-side end of the second outer side-sleeve strip 25) and (ii) a second proximal end fixed to the ankle support sleeve's interior surface nearer the ankle support sleeve's proximal end (i.e., an upper proximal fixation 53p toward the knee-side end of the second outer side-sleeve strip 25). In the exemplary PTTD-brace embodiment depicted in FIGS. 11-14, four upper second side-sleeve eyelets 59a, 59e, 59f, 59j are positioned at the ankle support sleeve's second side-sleeve section 24 between the upper lace's upper distal fixation 53d and the upper lace's upper proximal fixation 53p, and four upper first side-sleeve eyelets 57b, 57d, 57g, 57i are positioned at the ankle support sleeve's first side-sleeve section 22. As noted, the first outer side-sleeve strip 23 and the second outer side-sleeve strip 25 can function as eyestays (e.g., the four upper second side-sleeve eyelets 59a, 59e, 59f, 59j can be formed through second outer side-sleeve strip 25, and the four upper first side-sleeve eyelets 57b, 57d, 57g, 57i can be formed through the first outer side-sleeve strip 23).

As illustrated in FIGS. 11-14 in which the ends of upper lace 52 are fixed to the interior surface of the second side-sleeve section 24 (e.g., at the second outer side-sleeve strip 25 on the right side as viewed from the patient's perspective), pulling the upper lacing closure tab 54 away from the ankle support sleeve's first side-sleeve section 22 (and over and around the ankle support sleeve's second side-sleeve section 24) uniformly closes and tensions the ankle-brace boot 12 by drawing together the ankle support sleeve's first side-sleeve section 22 and ankle support sleeve's second side-sleeve section 24. As viewed from the patient's perspective, the upper lacing interconnection system 50 closes from left to right as shown in the exemplary arch-support brace of FIGS. 11-14.

As illustrated in the exemplary PTTD-brace embodiment depicted in FIGS. 11-14, the arch-support brace 10 includes a compression-enhancing lacing configuration that readily equilibrates (e.g., simultaneously evens) the tension in the upper lace 52 as the upper lacing closure tab 54 is extended from the ankle-brace boot 12. Near the middle of the arch-support brace 10, the upper lace 52 passes from its upper distal fixation 53d at the interior surface of the ankle support sleeve's second side-sleeve section 24 to and through an optional upper second outer distal eyelet 59a as depicted in FIG. 2.

With respect to FIGS. 11-14 as viewed from the patient's perspective, the ankle support sleeve's second side-sleeve section 24 is to the patient's right and the ankle support sleeve's first side-sleeve section 22 is to the patient's left. In one alternative embodiment (not shown), the distal end portion of the upper lace 52 can be fixed to the tongue 30 or another part of the ankle-brace boot 12 (e.g., an eyelet). In another alternative embodiment (not shown), the upper lace 52 is not immovably affixed to the ankle support sleeve 20 but rather knotted or otherwise configured (e.g., enlarged) so that the distal end portion of the upper lace 52 cannot pass through the upper second outer distal eyelet 59a or the like. For example, an end portion of the upper lace 52 can be doubled over, looped, or otherwise provided with any other suitable kind of obstruction, such as an enlargement (e.g., formed by deforming or melting), a whipping knot, a T-bar, or the like. In yet another alternative embodiment (not shown), the upper lace 52 can be fixed (e.g., stitched) to the exterior surface of the ankle support sleeve's second side-sleeve section 22, thereby rendering unnecessary the upper second outer distal eyelet 59a.

As illustrated in the exemplary ankle-brace embodiment depicted in FIG. 12, from the upper second outer distal eyelet 59a, the upper lace 52 passes to and through an upper first outer distal eyelet 57b, to and through an upper distal closure-tab eyelet 55c, to and through an upper first inner distal eyelet 57d, and to and through an upper second inner distal eyelet 59e. The upper lace 52 then passes lengthwise along the interior surface of the ankle support sleeve's second side-sleeve section 24 (e.g., along the interior surface of the second outer side-sleeve strip 25) from the upper second inner distal eyelet 59e to and through an upper second inner proximal eyelet 59f so that, between the upper second inner distal eyelet 59e and the upper second inner proximal eyelet 59f, the upper lace 52 is substantially parallel to the adjacent outer edge of the ankle support sleeve 20. The upper lace 52 then passes from the upper second inner proximal eyelet 59f to and through an upper first inner proximal eyelet 57g, to and through an upper proximal closure-tab eyelet 55h, to and through an upper first outer proximal eyelet 57i, to and through an optional upper second outer proximal eyelet 59j, and to an upper proximal fixation 53p at the interior surface of the ankle support sleeve's second side-sleeve section 24.

In one alternative embodiment (not shown), the proximal end portion of the upper lace 52 can be fixed to the tongue 30 or another part of the ankle-brace boot 12 (e.g., an eyelet). In another alternative embodiment (not shown), the upper lace 52 is not immovably affixed to the ankle support sleeve 20 but rather knotted or otherwise configured (e.g., enlarged) so that the proximal end portion of the upper lace 52 cannot pass through the upper second outer proximal eyelet 59j or the like. For example, an end portion of the upper lace 52 can be doubled over, looped, or otherwise provided with any other suitable kind of obstruction, such as an enlargement (e.g., formed by deforming or melting), a whipping knot, a T-bar, or the like. In yet another alternative embodiment (not shown), the upper lace 52 can be fixed (e.g., stitched) to the exterior surface of the ankle support sleeve's second side-sleeve section 24, thereby rendering unnecessary the upper second outer proximal eyelet 59j.

In ankle-brace embodiments in which an end portion of a lace is fixedly connected to an eyelet or otherwise restricted with respect to an eyelet (e.g., by knotting to restrict the end of the lace from passing through the eyelet), the eyelet may function as a mount rather than a lace-redirection mechanism.

The exemplary arch-support braces depicted in FIGS. 11-14 include both a lower lacing interconnection system 40 and an upper lacing interconnection system 50. Neither of these lacing interconnection systems 40, 50 fixedly attaches its single lace 42, 52 to the corresponding lacing closure tab 44, 54, either fully or partially. Rather, in these exemplary PTTD-brace embodiments, each lace 42, 52 may pass substantially freely through each of the corresponding closure-tab eyelets 45, 55. As depicted in FIGS. 1-6, each strand of lace 42, 52 connecting the ankle support sleeve 20 with a corresponding lacing closure tab 44, 54 is freely threaded through a corresponding closure-tab eyelet 45, 55. This “free-floating” closure-tab design enables the patient to readily equilibrate lace tension through the various eyelets positioned along the length of the ankle support sleeve 20 in a way that applies substantially consistent compression to the patient's lower leg and ankle. In this regard, each lace 42, 52 and its corresponding, moveably attached lacing closure tab 44, 54 wrap circumferentially around—rather than helically along—the patient's lower leg and ankle in a way that uniformly closes and tensions the ankle-brace boot 12.

In exemplary PTTD-brace embodiments such as depicted in FIG. 12, the lower lace 42, the upper lace 52, or both pass through one or more lacing channels, which may be secured to or otherwise formed in the tongue 30. For example, one or more lacing channels 62 may be positioned near the midpoint, near the distal end, and/or near the proximal end of a cushioned, stretchable tongue 30. The lower lace 42 may pass through a lower lacing channel 62 secured to the tongue 30 as the lower lace 42 passes from the lower first outer distal eyelet 47a to the lower second outer distal eyelet 49b, or the lower lace 42 may pass through a lower lacing channel 62 secured to the tongue 30 as the lower lace 42 passes from the lower second outer proximal eyelet 49i to the lower first outer proximal eyelet 47j. Similarly, the upper lace 52 may pass through an upper lacing channel 62 secured to the tongue 30 as the upper lace 52 passes from the upper second outer distal eyelet 59a to the upper first outer distal eyelet 57b (FIG. 12), or the upper lace 52 may pass through an upper lacing channel 62 secured to the tongue 30 as the upper lace 52 passes from the upper first outer proximal eyelet 57i to the upper second outer proximal eyelet 59j.

As will be appreciated by those having ordinary skill in the art, each lacing channel 62 should be positioned to facilitate passage of either the lower lace 42 or the upper lace 52 between the ankle support sleeve's first side-sleeve section 22 and the ankle support sleeve's second side-sleeve section 24 (e.g., between the first outer side-sleeve strip 23 and second outer side-sleeve strip 25). Positioning lacing channel(s) 62 at either the distal end or the proximal end of the tongue 30, or both the distal end and the proximal end of the tongue 30, helps to maintain the proper positioning of the tongue 30 against the patient's lower leg. In practice, securing a lace 42, 52 to the tongue 30 via one or more lacing channels 62 (e.g., at the distal end and/or the proximal end of a cushioned, stretchable tongue 30) reduces patient discomfort by preventing the lace 42, 52 from migrating beyond the end of the tongue 30 when the arch-support brace 10 is compressively and securely applied to the patient's ankle and lower leg.

As illustrated in the exemplary PTTD-brace embodiments depicted in FIGS. 11-14, hook-and-loop fasteners 65 (e.g., VELCRO-brand fasteners) are secured to the surfaces of the arch-support brace 10 to facilitate closure of the arch-support brace 10 to the patient's lower leg, ankle, and foot. For example, the ankle support sleeve 20, including the first side-sleeve section 22 and the second side-sleeve section 24, include a surficial fabric that functions as a loop fastener. Alternatively, loop-fasteners 66 (e.g., loop-fastener strip(s)) can be secured to the outer surfaces of the ankle support sleeve 20, typically at the ankle support sleeve's first side-sleeve section 22 and the ankle support sleeve's second side-sleeve section 24. Hook-fasteners 67 are provided elsewhere on the arch-support brace 10, such as on a surface of each lacing closure tab 44, 54.

As illustrated in FIGS. 1-6, a patient may secure the arch-support brace 10 to his ankle via one or more lacing closure mechanisms (e.g., the lower lacing interconnection system 40 and the upper lacing interconnection system 50). Wrapping the respective lacing closure tabs 44, 54 circumferentially around the ankle-brace boot 12 evenly closes and tensions the ankle-brace boot 12 by drawing together the ankle support sleeve's first side-sleeve section 22 and the ankle support sleeve's second side-sleeve section 24. Depending on the girth of the patient's lower leg, the hook-fasteners 67 on a surface of each lacing closure tab 44, 54 will engage loop-fasteners 66 positioned upon the respective outer surfaces of ankle support sleeve 20.

To supplement the present disclosure, this application incorporates entirely by reference the following commonly assigned patents and patent application publications: U.S. Pat. Nos. 5,067,486; 5,795,316; 7,651,472; 8,721,578; 8,808,215; 9,375,339; 9,393,146; 9,737,430; U.S. Patent Application Publication No. 2009/0112140 A1; U.S. Patent Application Publication No. 2014/0249460 A1; U.S. Patent Application Publication No. 2017/0143526 A1; U.S. Patent Application Publication No. 2017/0143530 A1; and U.S. Patent Application Publication No. 2018/0140451 A1.

In the specification and/or figures, typical embodiments of the PTTD-brace invention have been disclosed. The present PTTD-brace invention is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

1. A method of configuring an arch-support brace, comprising:
adjusting a position of an arch-support pad along a tibial-tendon support strap of the arch-support brace, wherein:
an ankle support sleeve of the arch-support brace comprises an inner side-sleeve section, an outer side-sleeve section, and a bottom-sleeve section connected to both of the first side-sleeve section and the second side-sleeve section,
the tibial-tendon support strap comprises a fixedly-attached end portion and an opposite end portion, and
the fixedly-attached end portion is directly fixedly attached to the outer side-sleeve section at a first position of attachment;
under-then-over wrapping the tibial-tendon support strap at least partially around the ankle support sleeve so that the tibial-tendon support strap becomes configured in a predetermined wrapped configuration wherein, in a direction along a length of the tibial-tendon support strap extending from the fixedly-attached end portion to the opposite end portion, the tibial-tendon support strap extends:
under the ankle support sleeve from the outer side-sleeve section to the inner side-sleeve section, and
then over the ankle support sleeve from the inner side-sleeve section to the outer side-sleeve section; and
releasably attaching an inner side of the opposite end portion of the tibial-tendon support strap to an outer side of the tibial-tendon support strap at a position of attachment that is laterally superposed with both the first position of attachment and the outer side-sleeve section, wherein the releasably attaching occurs while:
the tibial-tendon support strap is in the predetermined wrapped configuration, and
the arch-support pad is (i) engaged against the bottom-sleeve section of the ankle support sleeve, (ii) engaged against a portion of the tibial-tendon support strap, and (iii) positioned between the portion of the tibial-tendon support strap and the bottom-sleeve section of the ankle support sleeve.

2. The method according to claim 1, wherein the under-then-over wrapping of the tibial-tendon support strap is performed so that, in the direction along the length of the tibial-tendon support strap extending from the fixedly-attached end portion to the opposite end portion, the tibial-tendon support strap extends downwardly from the first position of attachment at an angle of inclination in a range of from about five degrees to about thirty degrees.

3. The method according to claim 2, wherein:
the predetermined wrapped configuration is a first predetermined wrapped configuration;
a stabilizing strap comprises a fixedly-attached end portion and an opposite end portion; and
the fixedly-attached end portion of the stabilizing strap is fixedly attached to the ankle support sleeve;
the method comprises over-then-under wrapping the stabilizing strap at least partially around the ankle support sleeve so that the stabilizing strap becomes configured in a second predetermined wrapped configuration wherein, in a direction along a length of the stabilizing strap extending from the fixedly-attached end portion of the stabilizing strap to the opposite end portion of the stabilizing strap, the stabilizing strap extends:
over the ankle support sleeve from the inner side-sleeve section to the outer side-sleeve section, and
then under the ankle support sleeve from the outer side-sleeve section to the inner side-sleeve section; and
releasably attaching an inner side of the opposite end portion of the stabilizing strap to the ankle support sleeve while both the tibial-tendon support strap is in the first predetermined wrapped configuration and the stabilizing strap is in the second predetermined wrapped configuration.

4. The method according to claim 3, wherein the over-then-under wrapping of the stabilizing strap is comprised of wrapping the stabilizing strap over the tibial-tendon support strap while the tibial-tendon support strap is in the first predetermined wrapped configuration.

5. The method according to claim 4, wherein:
the under-then-over wrapping of the tibial-tendon support strap occurs in a first direction, and
the over-then-under wrapping of the stabilizing strap occurs in a second direction that is opposite from the first direction.

6. The method according to claim 5, wherein the under-then-over wrapping of the tibial-tendon support strap is carried out so that, in the direction along the length of the tibial-tendon support strap extending from the fixedly-attached end portion of the tibial-tendon support strap to the opposite end portion of the tibial-tendon support strap, the tibial-tendon support strap extends:
under the ankle support sleeve from the outer side-sleeve section to the inner side-sleeve section,
then over the ankle support sleeve from the inner side-sleeve section to the outer side-sleeve section, and
then downwardly at an inclination along the outer side-sleeve section.

7. A method of configuring an arch-support brace, wherein the arch-support brace comprises an ankle support sleeve and a tibial-tendon support strap, the tibial-tendon support strap comprises a fixedly-attached end portion and an opposite end portion, the fixedly-attached end portion is fixedly attached to the ankle support sleeve, and the method comprises:
wrapping the tibial-tendon support strap at least partially around the ankle support sleeve so that the tibial-tendon support strap becomes configured in a predetermined wrapped configuration wherein, in a direction along a length of the tibial-tendon support strap extending from the fixedly-attached end portion to the opposite end portion, the tibial-tendon support strap extends:
under the ankle support sleeve from an outer side-sleeve section of the ankle support sleeve to an inner side-sleeve section of the ankle support sleeve, and
then over the ankle support sleeve from the inner side-sleeve section to the outer side-sleeve section; and
releasably attaching an inner side of the opposite end portion of the tibial-tendon support strap to the ankle support sleeve while the tibial-tendon support strap is in the predetermined wrapped configuration

8. The method according to claim 7, wherein the wrapping of the tibial-tendon support strap is comprised of encircling the ankle support sleeve with the tibial-tendon support strap, so that the tibial-tendon support strap encircles the ankle support sleeve in the predetermined wrapped configuration.

9. The method according to claim 7, comprising:
adjusting a position of an arch-support pad along the length of the tibial-tendon support strap; and
responsive to the adjusting of the position of the arch-support pad and the wrapping of the tibial-tendon support strap, the support pad is:
engaged against a bottom-sleeve section of the ankle support sleeve,
engaged against a portion of the tibial-tendon support strap, and
positioned between the portion of the tibial-tendon support strap and the bottom-sleeve section of the ankle support sleeve.

10. The method according to claim 7, wherein the releasably attaching of the inner side of the opposite end portion of the tibial-tendon support strap to the ankle support sleeve is comprised of releasably attaching the inner side of the opposite end portion of the tibial-tendon support strap to the outer side-sleeve section.

11. The method according to claim 10, wherein:
the fixedly-attached end portion is directly fixedly attached to the outer side-sleeve section,
the releasably attaching of the inner side of the opposite end portion of the tibial-tendon support strap to the outer side-sleeve section is comprised of directly releasably attaching the inner side of the opposite end portion of the tibial-tendon support strap to an outer side of the fixedly-attached end portion of the tibial-tendon support strap so that the inner side of the opposite end portion of the tibial-tendon support strap is indirectly releasably attached to the outer side-sleeve section.

12. The method according to claim 7, wherein:
the fixedly-attached end portion of the tibial-tendon support strap is fixedly attached to the outer side-sleeve section at a first position of attachment; and
the wrapping of the tibial-tendon support strap at least partially around the ankle support sleeve is comprised of wrapping so that, in the direction along the length of the tibial-tendon support strap extending from the fixedly-attached end portion to the opposite end portion, the tibial-tendon support strap extends downwardly from the first position of attachment at an angle of inclination in a range of from about five degrees to about thirty degrees.

13. The method according to claim 7, wherein:
the predetermined wrapped configuration is a first predetermined wrapped configuration;
a stabilizing strap comprises a fixedly-attached end portion and an opposite end portion; and
the fixedly-attached end portion of the stabilizing strap is fixedly attached to the ankle support sleeve;
the method comprises wrapping the stabilizing strap at least partially around the ankle support sleeve so that the stabilizing strap becomes configured in a second predetermined wrapped configuration wherein, in a direction along a length of the stabilizing strap extending from the fixedly-attached end portion of the stabilizing strap to the opposite end portion of the stabilizing strap, the stabilizing strap extends:
over the ankle support sleeve from the inner side-sleeve section to the outer side-sleeve section, and
then under the ankle support sleeve from the outer side-sleeve section to the inner side-sleeve section; and
releasably attaching an inner side of the opposite end portion of the stabilizing strap to the ankle support sleeve while both the tibial-tendon support strap is in the first predetermined wrapped configuration and the stabilizing strap is in the second predetermined wrapped configuration.

14. The method according to claim 13, wherein the wrapping of the stabilizing strap at least partially around the ankle support sleeve is comprised of wrapping the stabilizing strap over the tibial-tendon support strap while the tibial-tendon support strap is in the first predetermined wrapped configuration.

15. The method according to claim 13, wherein:
the wrapping of the tibial-tendon support strap is comprised of under-then-over wrapping, and
the wrapping of the stabilizing strap is comprised of over-then-under wrapping in a direction opposite from the under-then-over wrapping of the tibial-tendon support strap.

16. The method according to claim 15, wherein the under-then-over wrapping of the tibial-tendon support strap is carried out so that, in the direction along the length of the tibial-tendon support strap extending from the fixedly-attached end portion of the tibial-tendon support strap to the opposite end portion of the tibial-tendon support strap, the tibial-tendon support strap extends:
under the ankle support sleeve from the outer side-sleeve section to the inner side-sleeve section,
then over the ankle support sleeve from the inner side-sleeve section to the outer side-sleeve section, and
then downwardly at an inclination along the outer side-sleeve section.

17. An arch-support brace, comprising:
an ankle support sleeve comprising an inner side-sleeve section and an outer side-sleeve section;
a tibial-tendon support strap comprising a fixedly-attached end portion and an opposite end portion;
the fixedly-attached end portion being fixedly attached to the ankle support sleeve at a first position of attachment;
an inner side of the opposite end portion being releasably attached to the ankle support sleeve at a second position of attachment; and
the tibial-tendon support strap extending at least partially around the ankle support sleeve so that, in a direction along a length of the tibial-tendon support strap extending from the fixedly-attached end portion to the opposite end portion, the tibial-tendon support strap extends:
under the ankle support sleeve from the outer side-sleeve section to the inner side-sleeve section, and
then over the ankle support sleeve from the inner side-sleeve section to the outer side-sleeve section,
wherein the tibial-tendon support strap is configured to apply a force to assist a patient's tibial tendon when the arch-support brace is secured to the patient's foot.

18. The arch-support brace according to claim 17, wherein the fixedly-attached end portion of the tibial-tendon support strap is directly fixedly attached to the outer side-sleeve section.

19. The arch-support brace according to claim 17, wherein the tibial-tendon support strap extends at least partially around the ankle support sleeve so that, in the direction along the length of the tibial-tendon support strap extending from the fixedly-attached end portion to the opposite end portion, the tibial-tendon support strap extends:
under the ankle support sleeve from the outer side-sleeve section to the inner side-sleeve section,
then over the ankle support sleeve from the inner side-sleeve section to the outer side-sleeve section, and
then downwardly at an inclination along the outer side-sleeve section.

20. The arch-support brace according to claim 17, wherein the arch-support brace is configured so that, when the arch-support brace is secured to a patient's foot and ankle, the first position of attachment is laterally superposed with the patient's lateral malleolus.

21. The arch-support brace according to claim 20, wherein when the arch-support brace is secured to the patient's foot and ankle, the second position of attachment is laterally superposed with the patient's lateral malleolus.

22. The arch-support brace according to claim 17, wherein the inner side of the opposite end portion is releasably attached to the outer side-sleeve section.

23. The arch-support brace according to claim 22, wherein the inner side of the opposite end portion is directly releasably attached to an outer side of the fixedly-attached end portion so that the inner side of the opposite end portion is indirectly releasably attached to the outer side-sleeve section.

24. The arch-support brace according to claim 17, comprising an arch-support pad moveably connected to the tibial-tendon support strap to facilitate repositioning of the arch-support pad along the length of the tibial-tendon support strap.

25. The arch-support brace according to claim 24, wherein:
when the arch-support brace is secured to the patient's foot and ankle, an angle of inclination relative to vertical of a length of the tibial-tendon support strap extending from the first position of attachment to the patient's planted foot is configured in a manner that allows the fully and securely installed tibial-tendon support strap to lay flat while (i) the tibial-tendon support strap extends across an apex of the patient's medial arch and (ii) the arch-support pad engages the apex of the patient's medial arch; and
the angle of inclination is in a range of from about five degrees to about thirty degrees.
